SEQUENCE LISTING (33 sequences)

SEQ ID NO: 1 - DNA, 7 bp, Rat
tttgcat

SEQ ID NO: 2 - DNA, 30 bp, Rat
aaatattggtttgcataatcattgactgcc

SEQ ID NO: 3 - DNA, 7 bp, Rat
gggtacg

SEQ ID NO: 4 - DNA, 37 bp, Rat
aaatattggtttgcataatcattgactgcctactgag

SEQ ID NO: 5 - DNA, 30 bp, Rat
tttataaccaaacgtattagtaactgacgg

SEQ ID NO: 6 - DNA, 37 bp, Rat
tttataaccaaacgtaaatgtaactgacggatgactc

SEQ ID NO: 7 - DNA, 15 bp, Rat
ttggtttgcataatc

SEQ ID NO: 8 - DNA, 19 bp, Rat
tattggtttgcataatcat

SEQ ID NO: 9 - DNA, 19 bp, Rat
aaatgttttgcatgtgtta

SEQ ID NO: 10 - DNA, 19 bp, Mouse
tatgtctttgcataatgtt

SEQ ID NO: 11 - DNA, 19 bp, Human
caggggtttgcatggaccc

SEQ ID NO: 12 - DNA, 19 bp, Mouse
aaaagttttgcatgtcttt

SEQ ID NO: 13 - DNA, 19 bp, Mouse
atctgctttgcatggatct

SEQ ID NO: 14 - DNA, 19 bp, Human
tgtacctttgcatgtgttg

SEQ ID NO: 15 - DNA, 19 bp, Mouse
acgctttttgcattcccgc

SEQ ID NO: 16 - DNA, 19 bp, Rat
ctcctttttgcattttcct

SEQ ID NO: 17 - DNA, 19 bp, Human
cagcagtttgcatattttt

SEQ ID NO: 18 - DNA, 19 bp, Mouse
agactctttgcatctcagt

SEQ ID NO: 19 - DNA, 19 bp, Mouse
tacaggtttgcatcacgtt

SEQ ID NO: 20 - DNA, 8 bp, Rat
atttgcat

SEQ ID NO: 21 - DNA, 22 bp, Rat
actgaggataaggcagagttgc

SEQ ID NO: 22 - DNA, 21 bp, Rat
gagtcttgttcacctgcttgg

SEQ ID NO: 23 - DNA, 45 bp, Rat
gtcaatgattatgcaaaccaatatttgcgtcaggctagcctgacg

SEQ ID NO: 24 - DNA, 17 bp, Rat
tctcactgagtcctaaa

SEQ ID NO: 25 - DNA, 17 bp, Rat
agagtgactcaggattt

SEQ ID NO: 26 - DNA, 30 bp, Rat
gaagaaagtttaagatctccagaaagtttc

SEQ ID NO: 27 - DNA, 30 bp, Rat
cttctttcaaattctagaggtctttcaaag

SEQ ID NO: 28 - DNA, 30 bp, Rat
tactgagtccctggcgctattagacagcaa

SEQ ID NO: 29 - DNA, 30 bp, Rat
atgactcagggaccgcgataatctgtcgtt

SEQ ID NO: 30 - DNA, 30 bp, Rat
gaaaatagggacaggtgggtgaaaagttat

SEQ ID NO: 31 - DNA, 30 bp, Rat
cttttatccctgtccacccacttttcaata

SEQ ID NO: 32 - DNA, 22 bp, Rat
gactttcagatagttgggcaga

SEQ ID NO: 33 - DNA, 22 bp, Rat
ctgaaagtctatcaacccgtct

(19) United States
(12) Patent Application Publication
(10) Pub. No.: US 2003/0170736 A1
(43) Pub. Date: Sep. 11, 2003

(54) Methods and compositions for producing neural progenitor cells

(76) Inventor: Denes V. Agoston, Bethesda, MD (US)

Correspondence Address: MORRISON & FOERSTER LLP, 1650 TYSONS BOULEVARD, SUITE 300, MCLEAN, VA, 22102, US

(21) Appl. No.: 10/059,273

(22) Filed: Jan. 31, 2002

Related U.S. Application Data
(60) Provisional application No. 60/265,113, filed on Jan. 31, 2001.

(51) Int. Cl. G01N 33/53; G01N 33/567; C12N 5/08; C07K 16/40; C12N 15/87

(52) U.S. Cl. 435/7.2; 435/455; 435/368; 530/388.26

(57) ABSTRACT

The invention relates generally to methods and compositions for altering the differentiation status of cells such as stem and progenitor cells, and producing these cells for transplantation into mammals. The differentiation status of cells can be altered by contacting a nucleic acid decoy molecule to a mammalian cells and culturing the cell, whereby the differentiation status of the cell is altered. Pharmaceutical compositions of the invention are capable of entering a cell and binding to a protein in the cell and thereby altering a septamer function, a septamer-downstream function or a septamer-related function. The methods disclosed herein can be used in treating diseases by providing new cells to ameliorate symptoms of the disorder. Preferably, methods of the invention create homogeneous populations of progenitor and other cells, that can be administered to patients by transplantation. Diseases and disorders that can be treated in this fashion include, but are not limited to, CNS disorders, disorders of the lymphatic system, endothelial cell disorders, epithelial cell disorders, erythropoietic and hematopoietic diseases and disorders, neuro-degenerative disease, and traumatic brain injuries.

### RELATED APPLICATION

This application claims priority to U.S. provisional patent application Serial No. 60/265,113, filed Jan. 31, 2001, the entirety of which is hereby incorporated by reference.

### BACKGROUND OF THE INVENTION

1. Field of the Invention

The present invention generally relates to methods and compositions comprising nucleic acid decoys for altering the differentiation of cells, and in particular, stem and progenitor cells. The invention also relates to methods of treating diseases and to pharmaceutical compositions used in diagnosis and therapy of diseases such as neurological diseases and disorders of the CNS.

2. Description of the Background

Neurons and glial cells of the adult mammalian CNS are derived from multipotent progenitors that develop into neurons and glial cells (Brustle and McKay, 1996; Lillien, 1998; Pincus et al., 1998). At the level of transcriptional control, neurodifferentiation is envisaged as a cascade of spatiotemporally coordinated and cooperative interactions between regulatory DNA elements of neuron- or glial-specific genes and transcription factors that are differentially expressed in developing cells at progressive stages of neural development (Lemke, 1993). However, the identity of these transcription factors, as well as their coordinated interactions that must occur during development, is poorly understood because of the enormous complexity of the mammalian CNS. One approach to overcome this complexity is to search for mammalian homologs of genes identified in Drosophila or Caenorhabditis elegans (He and Rosenfeld, 1991). Despite some setbacks, for example, gcm (Kim et al., 1998), this strategy has been successful (Rubenstein and Beachy, 1998).

Another approach is to directly probe regulatory DNA fragments from genes that specify a neuronal or glial phenotype with nuclear proteins. Nuclear proteins isolated from distinct brain regions obtained at different developmental stages will include transcription factors that act at various stages of neural differentiation to specify a neuronal phenotype (Dobi et al., 1997). These transcription factors can be identified by the complexes that they form with their cognate DNA targets in electrophoretic mobility shift assays (EMSAs) and by DNA foot-printing. This approach can be used to identify the DNA motifs and their transcription factors whose interactions guide neural precursors into a given phenotype.

### SUMMARY OF THE INVENTION

The following summary and detailed description are exemplary of the scope and content of the invention. Additional embodiments can be devised or selected by one skilled in the art. Each of the references noted and listed under Appendix A, attached herewith, are specifically incorporated herein by reference. Each reference can also be used and relied on to make and use embodiments of the invention. Furthermore, reference texts like Ausubel et al. (2000) Current Protocols in Molecular Biology, John Wiley & Sons (specifically incorporated herein by reference in its entirety), can be relied on and used for known techniques in the art, including antibody production, cell culture and treatment, mRNA and protein assays for expression levels, and protein purification and isolation, for example.

Transfection of cis-element specific dsDNA, (“decoy DNA”) represents a new class of anti-gene strategy for gene transfer and transcriptional studies. One embodiment of the invention provides a nucleic acid decoy molecule that is designed to contain a binding site for a transcription factor of interest. Cells can take up nucleic acid decoy molecules at least up to about 50 to about 70 nucleotides long quite efficiently. Uptake can be increased using DNA delivery systems, such as cationic liquids or PEI for example. Upon internalization, the decoy nucleic acid molecules compete for binding to a nucleic acid binding protein preferably a transcription factor and more preferably an endogenous transcription factor. As a consequence, the endogenous DNA motif is measurably reduced in its ability to bind protein(s), resulting in the altered expression of genes that harbor the DNA motif and/or functional changes, which can be detected by measuring the abundance of mRNA or by observing alterations in cellular phenotype or physiology. Similar to the antisense technology's “loss-of function” approach, nucleic acid decoy can also be “loss-of-function” approach, however it affects gene function at pre-transcriptional and transcriptional level.

Application of a nucleic acid molecular decoy strategy permits not only highly specific functional studies but also therapeutic interventions. DNA decoy strategy has been successfully used for blocking viral proliferation, tumor growth, apoptosis, muscle differentiation and modulating the neuronal response to various stimuli. Recently, a 2000 patient size clinical trial has been conducted using dsDNA decoy to block vascular smooth muscle cell proliferation in venal grafts prior to bypass surgery. Thus, embodiments of the invention can be used for diagnostic and therapeutic methods involving neural cells and diseases or conditions of the CNS.

The enkephalin (ENK) gene can be used as model system for defining the transcriptional events underlying neural differentiation. Enkephalinergic neurons mediate social behavior, reward and aggression (Martin et al., 1991; Konig et al., 1996). Because of their spatially contrasting distribution they represent a convenient model system to explore transcriptional regulation of phenotypic differentiation in neural progenitors. Interactions between nuclear proteins derived from early embryonic brain [embryonic days 12-14 (E12-E14)] and DNA elements from the ENK gene can be used to identify DNA motifs that are involved in controlling general differentiation steps, such as the entry of progenitors into the neuronal or glial lineage. DNA fragments used in initial studies (Dobi et al., 1997) encompassed the entire regulatory region of the rodent ENK gene (Rosen et al., 1984; Dobi et al., 1995a; Agoston et al., 1998).

Screening studies identified a novel DNA element (septamer), which, in addition to the ENK gene, is also present on the regulatory regions of other neuron- and glial-specific genes. The invention, in one embodiment, discloses advantage of the septamer element as a binding site for developmentally expressed nuclear proteins. In another embodiment, the invention comprises septamer element recognition factors, termed sept proteins, and complexes formed with sept proteins and septamer. Exemplary sept proteins are the 23 kDa g-sept, and the 29 kDa n-sept derived from primary CNS cultures. These proteins can be purified using DNA-affinity chromatography with septamer-containing DNA. The sept proteins can be used in diagnostic assays. For example, g-sept levels are increased in proliferating glioma cells, indicating that increased g-sept levels are a marker for astroglioma tumors and related neural cell tumors. Altering or regulating septamer function, septamer downstream function or otherwise related function, by introducing specific competitor DNA molecules into differentiating neural cultures results in altering regulation of the expression of neuronal- and glial-specific genes. Thus, the methods and compositions of the invention can be used to produce novel cells and these cells are an important aspect of the invention.

The present invention relates to methods and compositions for altering the differentiation status of and producing progenitor cells, more specifically, a population of progenitor cells. One embodiment of the invention provides a method for altering the differentiation of progenitor cell of a mammal, comprising contacting a nucleic acid molecule, which may be DNA, RNA or PNA, comprising the sequence set forth in SEQ ID NO:1 (5′-TTTGCAT-3′) or SEQ ID NO:2 (5′-AAATATTGGTTTGCATAATCATTGACTGCC-3′) to the cell, wherein the nucleic acid is capable of entering the cell; and culturing the cell, whereby the cell differentiates into a progenitor cell. More specifically the sequence may contains a base other than A, U or a derivative thereof, at the position immediately 5′ to the sequence of SEQ ID NO:1. In another embodiment, there is provided a cell produced by the method described herein.

Another embodiment of the invention provides a method for isolating a nuclear protein comprising preparing a nucleic acid molecule comprising the sequence set forth in SEQ ID NO:1 or SEQ ID NO:2; optionally linking the nucleic acid to a solid support, allowing cell or tissue extracts to contact the nucleic acid; and separating the protein bound to the nucleic acid from other components of the extract.

Another embodiment of the invention provides an antibody that specifically binds to an isolated complex comprising: a decoy molecule containing SEQ ID NO:1 or SEQ ID NO:2; and a nucleic acid binding protein which may be, but is not limited to, a p-sept protein, a g-sept protein, or an n-sept protein, or binding fragments or combinations thereof.

Another embodiment of the invention provides a diagnostic kit comprising an antibody or antibody fragment that specifically bind directly or indirectly to a differentiation marker. Suitable markers include, preferably, a p-sept, g-sept, n-sept, nestin or cyc D2 protein.

Another embodiment of the invention provides a method of identifying protein-nucleic acid interaction comprising: contacting cells or cell proteins with a nucleic acid, preferably, SEQ ID NO:1; and identifying proteins that specifically bind to the nucleic acid. More specifically the sequence may contains a base other than A, U or a derivative thereof, at the position immediately 5′ to the sequence of SEQ ID NO:1.

Another embodiment of the invention provides a method for producing a progenitor cell or cell line comprising: contacting a nucleic acid molecule, preferably, SEQ ID NO:1 or SEQ ID NO:2, to the cell, wherein the nucleic acid molecule is capable of entering the cell; and culturing the cells, thereby altering the differentiation status of the progenitor cell. Preferably, the cell differentiates into a progenitor cell and more preferably a neural progenitor cell. More specifically the sequence may contains a base other than A, U or a derivative thereof, at the position immediately 5′ to the sequence of SEQ ID NO:1. In another embodiment, there is provided a cell and cell line produced by the method described herein.

Another embodiment of the invention provides methods and compositions for altering the differentiation status cells to treat or ameliorate a disease or disorder. According to these methods, a progenitor or stem cell is alter to differentiate into any of various cell types, including but not limited to, cardiac, endothelial, epithelial, erythropoietic, glial, hematopoietic, lymphatic, neural (which may be peripheral or central), or any of a variety of additional cell types useful to those of ordinary skill in the art. The invention further relates to the cells and cell lines produced by these methods.

Another embodiment of the invention provides a method of treating a disease comprising: contacting a nucleic acid molecule comprising the sequence set forth in SEQ ID NO:1 or SEQ ID NO:2 to a cell preferably generating a homogeneous population of cells for transplantation. Treated cells may be administered to a patient after treatment or treatment may involve direct in vivo administration of the nucleic acid molecule to the patient. Diseases that can be treated preferably include CNS disorders, neuro-degenerative disease, and traumatic brain injuries. Treatment can ameliorates one or more symptoms of the disease.

Another embodiment of the invention provides complexes that comprise a nucleic acid decoy molecule such as, for example, SEQ ID NO:1 or SEQ ID NO:2; and a nuclear protein such as, for example, p-sept, g-sept, or n-sept protein, which have molecular masses of approximately 16 kDa, 23 kDa, or 29 kDa, respectively.

Another embodiment of the invention provides methods for detecting differentiation status of a progenitor or a stem cell, for example, by determining one or more of P-, N-, or G-protein bands, or by determining the presence, absence, or simply level of differentiation markers such as, for example, the expression levels of p-sept, g-sept, n-sept, nestin, or Cyclin D2.

Another embodiment of the invention provides a pharmaceutical composition comprising an isolated nucleic acid, for example, SEQ ID NO:1 or SEQ ID NO:2, wherein the DNA is capable of entering a cell and altering a physiological function of the cell. Preferably, the physiological function altered is the differentiation status of the cell. Preferably, entering the cell alters septamer function, downstream functions associated with septamer binding, or otherwise related activities. Preferably the cells are primary differentiating cells or neural cells wherein the decoy molecule can be used in therapeutics to treat CNS disorders such as, for example, neuro-degenerative diseases or traumatic brain injuries. More specifically the sequence may contains a base other than A at the position immediately 5′ to the sequence of SEQ ID NO:1.

Further features, objects, and advantages of the present invention are apparent in the claims and the detailed description that follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE DRAWINGS

FIG. 1. FIG. 1 depicts gels of electrophoretic mobility shift assay (EMSA) and footprinting assays, which show that nuclear proteins from embryonic brain bind to septamer (protein-DNA interaction).

FIG. 2. FIG. 2 shows septamer-binding proteins are associated with different cellular population in the developing rat CNS.

FIG. 3. FIG. 3 shows the effect of blocking septamer function in differentiating neural cultures by introducing exogenous competitor DNA molecule.

FIG. 4. FIG. 4 shows the identification of septamer-binding proteins by UV cross-linking.

FIG. 5. FIG. 5 depicts distinct bending effects of the various septamer binding protein complexes.

FIG. 6. FIG. 6 depicting a DNA competition assay using competitor DNA molecule the synthetic DNA fragment rENK-496;-467, which shows that the mutated fragment failed to bind nuclear proteins.

### DESCRIPTION OF THE INVENTION

Differentiation of progenitor or stem cells into neurons and glial is regulated by interactions between regulatory DNA elements of neuron- and glial-specific genes and transcription factors that are differentially expressed by progenitors at progressive stages of neural development. A novel DNA regulatory element TTTGCAT (SEQ ID NO:1), referred to as a septamer, is present on a number of neuronal and glial cell genes, including enkephalin (ENK), neuronal cell adhesion molecule, neurofilament of 68 kDa (NF68), growth-associated protein of 43 kDa, glial high-affinity glutamine transporter, and tyrosine hydroxylase, for example. This invention provides compositions of nucleic acids capable of blocking septamer function or activities associated with downstream septamer activity as competitor nucleic acid in primary differentiating cells cultures, for example neural cultures, wherein the decoy molecule and methods of using them to alter differentiation of the cells. This invention provides methods of altering differentiation status of cells, preferably neural progenitor cells, and generating a staged (homogeneous) population of cells, preferably progenitor cells, for transplantation or other purposes known to those of ordinary skill. The invention therefore provides compositions and methods for diagnostic and therapeutic use of nucleic acid regulatory elements, preferably elements that comprise the sequence of SEQ ID NO:1 or SEQ ID NO:2. Useful application include, but not limited to. cardiac diseases, epithelial cell transplantation, erythropoietic disorders, lymphatic disorders, neuro-degenerative diseases and traumatic brain injury. Analysis of the mRNA levels of ENK, NF68, and glial fibrillary acidic protein in the cells produced by the inventive methods shows a decrease by 50-80% after treatment with competitor nucleic acid, whereas no effect was seen using a control nucleic acid. Analysis of nestin mRNA and protein levels in the resulting cells demonstrates that functional progenitor or stem cell cultures are produced. These cells can be used to test for neural and glial differentiation activity or used in, preferably, transplantation methods.

While not bound to any specific theory, use of the invention indicates that septamer elements may serve as binding sites for lineage-specific multimeric complexes assembled from three distinct nuclear proteins. Progenitors express a 16 kDa protein (p-sept), which binds to nucleic acid as a homodimer (detected as the 32 kDa P-band). Cells that entered the neuronal lineage express an additional 29 kDa protein (n-sept) that binds to the homodimerized p-sept, and together they form a 62 kDa multimer (detected as N-band). Cells that entered the glial lineage express a distinct 23 kDa protein (g-sept), which along with the homodimerized p-sept form a 56 kDa multimer (observed as G-band). The binding of the distinct protein complexes (P, G, and N) to the septamer site causes a lineage-specific NUCLEIC ACID bending (P=53°; G=72°; and N=90°), which may contribute to the regulatory effect of the septamer interaction. Thus, the invention also comprises septamer-binding proteins and complexes, which represent novel protein-DNA interactions that allow regulation of neuroglial differentiation in the developing mammalian CNS.

The Septamer Motif is the Binding Site for Nuclear Proteins of the Developing Brain:

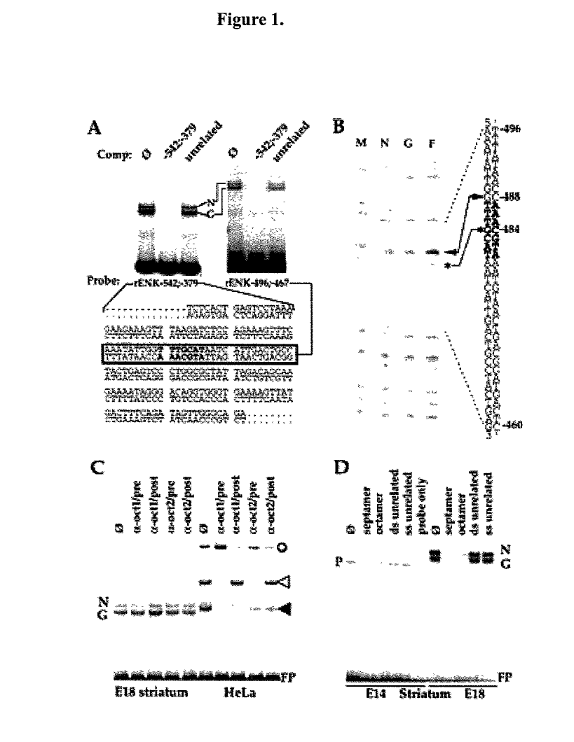

Referring to FIG. 1, gel pictures depict electrophoretic mobility and footprinting that nuclear proteins from embryonic brain bind to septamer (protein-DNA interaction). 1A: EMSA with nuclear extract from E17 striatum and either the entire 163 bp (rENK-542;-379) fragment or the 30 bp septamer-containing subfragment (reNK-496;-467) as radiolabeled probes. The cold competitors were either the rENK-542;-379 fragment or unrelated dsDNA. The sequence of the DNA probe rENK-542;-379 containing the septamer motif (in boldface) is depicted. The box marks the position and sequence of the 30 bp synthetic DNA oligonucleotide related to rENK-542;-379. Each probe displays a similar pattern of specific protein-DNA complexes: two closely spaced complexes, with the lower-mobility band expressed in lower amounts. The difference in the relative mobilities of complexes formed from the two probes is the result of the differences in length between the two probes (163 vs 30 bp). φ, No competitor; FP, indicates free probe. 1B: DNA methylation interference footprinting of rENK-542;-379 with nuclear extract for E18 striatum identifies the septamer motif (boldface) as the binding site. The G nucleotide positioned at 484 showed interference (asterisk), whereas no interference was seen at the G residue at 488 (arrow). M, G reaction marker; N, neuron-specific complex; G, glial-specific complex; F, free probe. 1C. Antibody supershift assay with nuclear extracts from E18 striatum and HeLa cells and rENK-542;-379 as probe. Nuclear proteins were preincubated with the antibodies overnight before addition of the probe (pre), or antibodies were added after formation of the DNA-protein complex (post). As a positive control, nuclear extracts derived from HeLa cells were used. HeLa cells express both Oct-1 and Oct-2-binding proteins, which form complexes of distinct mobilities (arrow, open arrowhead). Preincubation of the nuclear extracts with Oct-1 or Oct-2 antibodies (pre) resulted in a dramatically altered mobility of the complexes (supershift, marked by circle). 1D: The P complex present in E14 striatum can only be competed with septamer but not with octamer sequence. The N and the G complexes present in E18 striatum could, however, be competed by septamer as well as to some extent by the octamer sequence. Unrelated double-stranded (ds) and single-stranded (ss) DNA did not compete. FP, Free probe.

The initial screening for specific protein-DNA interactions using the PCR fragment rENK-542;-379 corresponding to the 5′ regulatory region of the rENK gene and nuclear extract derived from E18 striatum showed specific and abundant DNA-protein duplexes marked as N- and G-bands (See FIG. 1A). Toward the identification of the binding site, synthetic subfragments of rENK-542;-379 were tested in EMSA, and rENK-496;-467 formed a doublet that could be specifically competed only by excess original fragment (rENK-542;-379), but not by unrelated DNA.

To determine the site of base-specific interactions, DNA methylation interference footprinting was performed (See FIG. 1B). Footprinting showed a strong specific interaction between the binding proteins and the internal G-nucleotide at position −484 of TTGG⁴⁸⁸TTTG⁴⁸⁴CATAATC, (SEQ ID NO:7, underlined sequence here is septamer and boldface in FIG. 1B). Very importantly however, the 5′ purine base at position −488 was not involved in DNA-protein interactions, unlike the case for octamer-binding proteins. Because there was no base-specific interaction at the 5′ purine base, the nucleic acid element is named “septamer” motif.

Because of the sequence similarities between octamer nucleic acid elements and the septamer motif, it is anticipated that Oct-1 polyclonal antibody would recognize the septamer-binding proteins forming the N- and G-bands (See FIG. 1A), and this recognition would result in the supershifting of the formed DNA-protein complexes. Neither the anti-Oct-1 nor anti-Oct-2 antibodies recognized the nuclear proteins in the N and G complexes (See FIG. 1C). This experiment shows that proteins that bind to the septamer motif are distinct from the known proteins that bind to the octamer-element. In control experiments, the presence of anti-Oct-1 or anti-Oct-2 antibodies resulted in supershifted complexes when nuclear extracts from HeLa cells were tested (See FIG. 1C).

Competition assays using the rENK-496;-467 synthetic fragment that contains the septamer site or a nucleic acid fragment of equal length that contained the consensus oct-1 site as competitors (See FIG. 1D) were performed. These experiments show that the early, P-band (See below) was highly specific to the septamer motif. Binding was eliminated when the septamer-containing nucleic acid competitor was used. The presence of excess octamer competitor did not affect binding. The N- and G-bands detected in E18 striatum were also successfully competed with septamer nucleic acid. However, a low degree of competition was also observed with octamer-containing dsDNA.

The Different Septamer Complexes are Associated With Distinct Cellular Phenotypes:

To obtain the precise spatiotemporal distribution of the septamer-binding proteins, nuclear proteins derived from various brain regions were probed at distinct stages of neurodifferentiation with rENK-496;-467 (for sequence, see FIG. 1A). This screening revealed an interesting spatiotemporal distribution of the three previously observed protein-DNA complexes P, N, and G (See FIG. 1D).

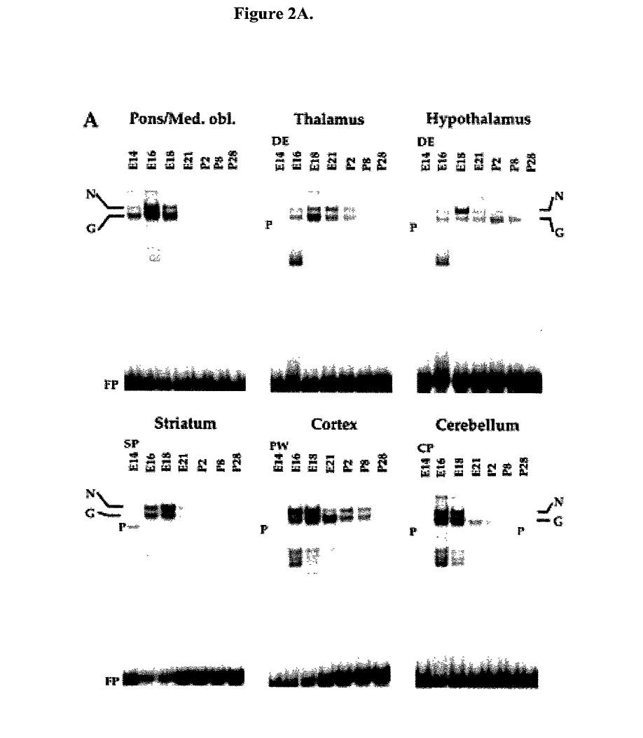

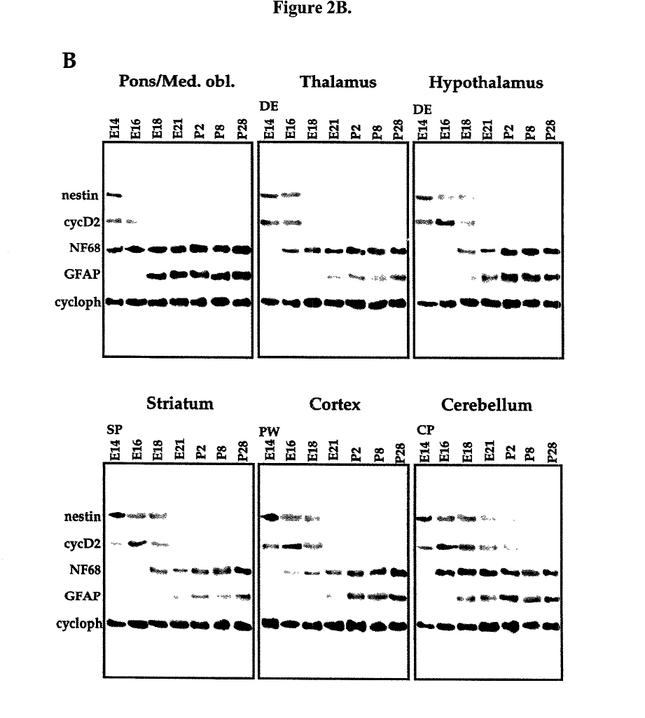

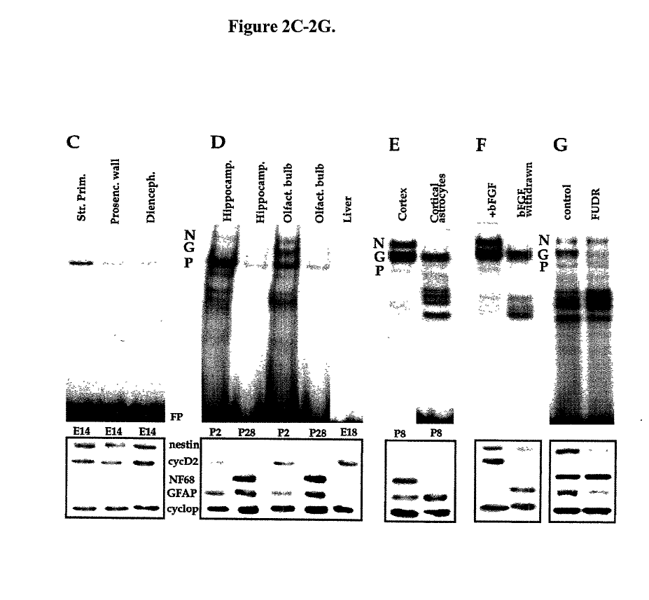

Referring to FIG. 2, shows septamer-binding proteins are associated with different cellular population in the developing rat CNS. 2A: Spatiotemporal distribution of septamer-binding proteins using the rENK-496;-467 fragment as probe in various brain regions between E14 and P28 by EMSA. P indicates the formation of a distinct mobility at early stages of neurodevelopment marking the generation of neuroepithelial progenitor cells. N indicates a complex whose formation coincides with neurogenesis, and G band indicates a complex whose formation coincides with gliogenesis; DE, diencephalon; SP, striatal primordium; PW, prosencephalic wall; CP, cerebellar primordium; Cortex, frontoparietal cortex. 2B: PCR amplicons indicating the abundance of selected markers as measured by RT-PCR; Cyclop, cyclophilin. 2C. P complex is formed in all primordial regions of the forebrain (striatal primordium, prosencephalic wall, and diencephalon), which all express nestin and cyclin D2 mRNA (bottom panels). 2D: Hippocampus and olfactory bulb contain the P-band both in early postnatal age (P2) and in adult (P28). Liver does not contain any of the septamer-binding proteins. 2E: Cultured primary cortical astrocytes contain only G-band, whereas the age-equivalent cortex has both N- and G-bands. 2F: EMSA of nuclear proteins derived from a striatal progenitor cell culture 6 d after mitogen treatment (+bFGF) shows the presence of all three bands, but 3 d after the mitogen was removed (bFGF withdrawn), only the G-band can be detected. The high-mobility bands were primarily detected when the abundant G-band was also seen. 2G: EMSA of nuclear proteins derived from E16 rat forebrain cultures grown for 2 d in the absence or in the presence of the mitotic inhibitor FUDR. Substantially decreased G-band and GFAP mRNA levels were measured in the FUDR-treated cultures, whereas N-band and NF68 mRNA remained unchanged. 2C-2G, bottom panels, Abundance of markers as in 2D.

The single, medium-mobility band marked as P that was detected in the striatal primordium at E14 was also seen in all tested brain regions except the pons and medulla oblongata at E14 (See FIG. 2A). Subsequent analysis, however, showed the presence of the P-band in E10-E12 rhombencephalon. RT-PCR analysis of the corresponding cytoplasmic supernatants demonstrated the expression of nestin and cycD2 mRNA in these brain regions, suggesting the presence of proliferating neural progenitors (See FIG. 2B). Neither NF68 nor GFAP mRNA, the markers for differentiated neurons and astroglia, was detected at this stage. Because of the expression of nestin and cycD2 mRNA and because the spatiotemporal pattern of P-band correlated with the generation of neural progenitors in the various brain primordia, this complex is designated “P” for progenitors. In the developing brain, the P-band was not detected after E14 except in the cerebellum, where the P-band reappeared between P2 and P8 (See FIG. 2A). The P-band was not detected in any brain regions of the adult brain except the olfactory bulb and the hippocampus (See FIG. 2D).

After E14, the lower-mobility double band (See FIG. 1D, marked as N, G) was also detected in all other brain regions tested (See FIG. 2A). Again, this doublet was observed in the ontogenetically earliest region, the pons and medulla oblongata, at E14 when other brain primordia (diencephalon, striatal primordium, and telencephalon) contained only the P-band (See FIGS. 2A, C). However, the spatio-temporal pattern of appearance and disappearance of the N- and G-bands closely resembled the developmental appearance of developing neurons and Gila, which varies among ontogenetically distinct brain regions (See FIG. 2A). For example, in the striatum, the most abundant N-band was observed at E18, which is the peak activity of neurogenesis (Bayer and Altman, 1995; See FIG. 2A). In the developing cerebral cortex, the N-band was most abundant between E16 and E18, but it remained detectable at early post-natal ages, consistent with a longer period of neurogenesis in this brain region. There were no detectable complexes in the adult (>P28) cerebral cortex or any other brain regions. RT-PCR analysis of the gene expression pattern showed that the decrease and disappearance of nestin mRNA expression and the appearance of the neuronal marker NF68 mRNA corresponded to the developmental appearance of the N-band in the various brain regions (See FIGS. 2A,B). Similarly, the spatiotemporal appearance of the G-band preceded the developmental appearance of GFAP mRNA, the marker for astroglia (See FIGS. 2A,B).

These observations indicate that the appearance of N-band marks an early developmental event for neuronal precursors, such as the entry of neural progenitors into the neuronal lineage or an early stage of neuronal differentiation. The appearance of the G-band also marks the entry of progenitors into the glial lineage or an early stage of glial differentiation. In addition to the N- and G-bands, high-mobility complexes were frequently observed in several brain regions at E16 (See FIG. 2A). These high-mobility complexes, which were also detected in astrocytic cultures (See FIG. 2E) and were enhanced after mitogen withdrawal of striatal cultures (See FIG. 2E), probably consist of dissociated and cleaved components of the septamer multimers (See below).

The association of P-, N-, and G-bands with distinct developmental stages was further investigated by using various cell culture systems. The presence of the various (P-, N-, and G-) bands in microdissected cerebral cortex at P8 to age-related cortical astrocytes in culture was also tested. Cerebral cortex contained both N and G complexes; however, only the G-band was detected in age-equivalent cortical astrocyte cultures (See FIG. 2E). In accord with the EMSA results, RT-PCR analysis demonstrated the expression of GFAP mRNA in astrocytic cultures whereas both GFAP and NF68 mRNA were detected in P8 cerebral cortex (See FIG. 2E). None of the three complexes (P-, N-, and G-bands) was observed in any peripheral tissues at any ages tested (See FIG. 2D).

Striatal culture system, developed by Johe et al. (1996), was also used. The culture originated from E16 striatum and was treated with the mitogen bFGF for 6 d. When these cultures were analyzed at the end of bFGF expansion (corresponding to E22 in vivo), the P-band as well as abundant N- and G-bands were detected (See FIG. 2F). After withdrawal of mitogen, all cells differentiated into astrocytes, as indicated by the abundant G-band and GFAP mRNA. P-band, N-band, and NF68 mRNA were undetectable; however, a low amount of nestin mRNA was detected (See FIG. 2F).

A positive association of the N-band with differentiating neurons was also established by taking advantage of the distinct onset of neurogenesis and gliogenesis in the developing CNS. Because blocking cellular proliferation after the majority of neurons are postmitotic but gliogenesis is ongoing should reduce G-band and GFAP mRNA levels but should leave N-band and NF68 mRNA relatively unchanged, developing neural cultures derived from E15 rat forebrain were treated with FUDR starting at 1 DIV (E16) for 2 d. Blocking cellular proliferation within this developmental period resulted in a substantial decrease in the intensity of the G-band complex on EMSA and the GFAP mRNA by RT-PCR, whereas the intensities of N-band and NF68 mRNA remained virtually unaffected (See FIG. 2G). These results further support the hypothesis that N- and G-bands are associated with differentiating neurons and Glial cells, respectively.

Detecting Changes in Differentiation Status of a Cell:

The Septamer Motif is Present on Various Neuronal- and Glial-Specific Genes:

The relative abundance and the developmentally regulated appearance of the P-, N-, and G-complexes in the developing rat CNS suggested that the septamer motif may be a nucleic acid regulatory element central to neural differentiation, and as such, it should be present within the regulatory regions of neuronal- and glial-specific genes. Searching the database (GenBank) for genes containing the septamer motif confirmed this assumption. The septamer motif was found on the 5′ regulatory regions of many neuronal- or glial-specific genes. These genes include substance P/neurokinin, NF68, growth-associated protein of 43 kDa (GAP-43), neuron cell adhesion molecule, and glial high-affinity glutamate transporter and vimentin (See Table 1).

TABLE 1
Genes with septamer element.

| GENE | SEQ ID NO: | SEQUENCE | ACCESSION NO. |
|---|---|---|---|
| Enkephalin^{a} | SEQ ID NO:8 | TATTGGTTTGCATAATCAT | X59136 |
| Substance P^{a} | SEQ ID NO:9 | AAATGTTTTGCATGTGTTA | L07328 |
| VIP^{b} | SEQ ID NO:10 | TATGTCTTTGCATAATGTT | X74297 |
| Tyrosine hydroxylase^{c} | SEQ ID NO:11 | CAGGGGTTTGCATGGACCC | M18114 |
| Neurofilament 68 kD^{b} | SEQ ID NO:12 | AAAAGTTTTGCATGTCTTT | U80021 |
| Necdin^{b} | SEQ ID NO:13 | ATCTGCTTTGCATGGATCT | D76440 |
| Synapsin I^{c} | SEQ ID NO:14 | TGTACCTTTGCATGTGTTG | M55301 |
| (m1) muscarinic | | | |
| ACh receptor^{b} | SEQ ID NO:15 | ACGCTTTTTGCATTCCCGC | AJ02973 |
| GAP-43^{a} | SEQ ID NO:16 | CTCCTTTTTGCATTTTCCT | S71492 |
| N-CAM^{c} | SEQ ID NO:17 | CAGCAGTTTGCATATTTTT | X53243 |
| glial high-affinity | | | |
| glutamate transporter^{b} | SEQ ID NO:18 | AGACTCTTTGCATCTCAGT | D63816 |
| vimentin^{b} | SEQ ID NO:19 | TACAGGTTTGCATCACGTT | D50805 |
| Septamer sequences within the 5′ regulatory region of genes are in boldface; | | | |
| the 5′ base is underlined. | | | |
| N-CAM, Neuronal cell adhesion molecule; | | | |
| VIP, vasoactive intestinal peptide. | | | |
| Accession numbers are from GenBank. | | | |
| ^{a}Rat; | | | |
| ^{b}mouse; | | | |
| ^{c}human | | | |

Altering the Binding of Septamer-Binding Proteins Alters Gene Expression:

To gain insight as to the possible function of the septamer motif and its binding proteins, a competitor double-stranded dsDNA molecule is used to decoy septamer proteins from their endogenous dsDNA binding sites. The decoy approach has been successfully used in numerous other systems and permits functional studies before the cloning of the DNA-binding protein or transcription factor of interest (Morishita et al., 1998). The invention provides the first use of molecular decoy to affect differentiation of neuronal and glial cells. Differentiating striatal cultures at E18 were transfected with the competitor dsDNA. The competitor molecule comprises the septamer motif (rENK-496;-467; for sequence information, see FIG. 1A). The control dsDNA contained a transversion mutant (purine to pyrimidine substitution) of the septamer motif (septamer^{mut}) in which the purine to pyrimidine substitution resulted in the loss of protein binding as tested by EMSA. The relative abundance of neuronal and glial markers (ENK, NF68, and GFAP) was decreased in cultures after transfection with the septamer motif-containing nucleic acid (See FIG. 3). ENK mRNA levels decreased by 80%, whereas NF68 and GFAP mRNA levels were ˜60% lower than in cultures transfected with septamer^{mut}. The abundance of cyclophilin mRNA did not changed significantly. There were no significant difference between cultures that were transfected with the septamer^{mut} dsDNA and nontransfected controls. The effects were highly reproducible (n=6).

Referring to FIG. 3, shows the effect of blocking septamer function in differentiating neural cultures by introducing exogenous competitor nucleic acid molecule. FIG. 3 shows results of RT-PCR analysis of gene expression patterns after the introduction of rENK-496;-467 as competitor dsDNA (septamer, solid bar) and a mutated control dsDNA (septamer^{mut}, hatched bars). cycloph, Cyclophilin. Values are expressed as percentage of the relative intensities obtained from control cultures that did not receive dsDNA. Error bars indicate SEM (n 6).

Lineage-Specific Protein Components of the Septamer-Binding Complex:

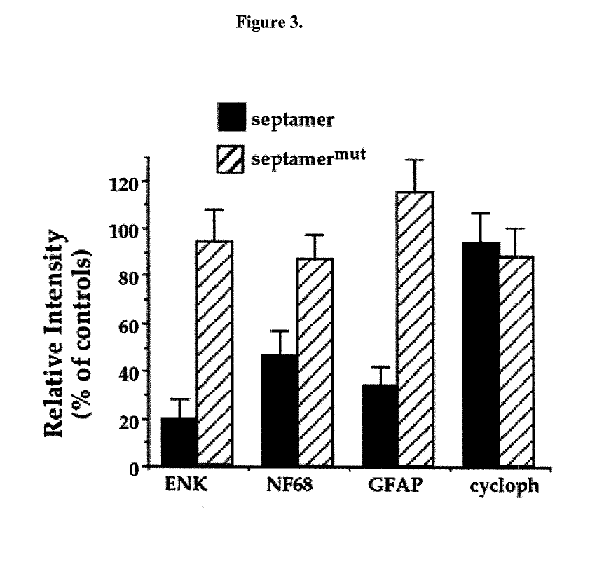

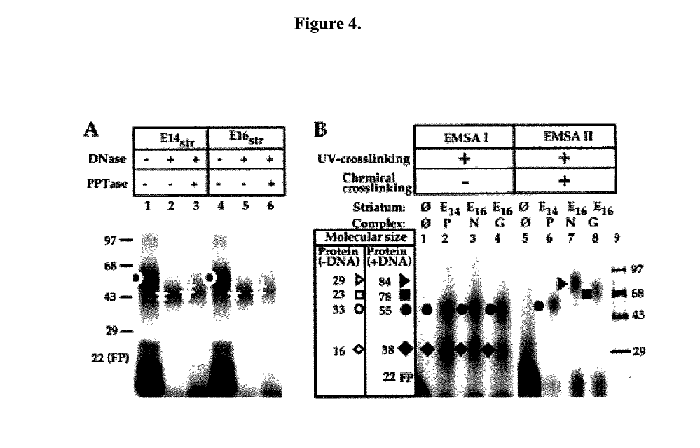

To identify the proteins that bind to the septamer motif and form the P-, N-, and G-bands, both solution- and EMSA-mediated (two-dimensional) UV cross-linking experiments were performed with and without simultaneous chemical cross-linking reactions. A dsDNA probe containing the septamer motif (rENK-496;-467) was UV cross-linked to nuclear proteins derived from E14 or E16 striatum. By EMSA, only the P-band was observed at E14, whereas at E16 no P-band but both N- and G-bands were detected (See FIGS. 2A,C). After UV cross-linking, however, there were no differences seen in the size of the protein-DNA complex derived form the E14 or E16 striatum (See FIG. 4A; compare lanes 1, 4). After deducting the size of the dsDNA probe, the size of the DNA-binding proteins was found to be 32 kDa in both E14 and E16 striatum. In addition to the identical size, incubation with DNase I and/or dephosphorylation of cross-linked proteins from E14 and E16 striatum resulted in identical changes in gel electrophoretic mobilities (See FIG. 4A; lanes 2, 5, 3, 6). These observations suggested that the core DNA binding protein may be identical in all three complexes (P, N, and G).

Referring to FIG. 4, shows the identification of septamer-binding proteins by UV cross-linking. 4A: Radioactively labeled rENK-496;-467 probe was combined with nuclear extracts from E14 and E16 striatum and was irradiated in solution with UV light. One-third of the cross-linked DNA-protein complex was analyzed without further treatments (lanes 1, 4); one-third was additionally incubated with DNase I (lanes 2, 5); and one-third of the cross-linked mixture was incubated with both DNase I and protein phosphatases (PPTase; lanes 3, 6). The complexes were analyzed on 4-20% SDS-polyacrylamide gel. A filled circle indicates the UV cross-linked protein-DNA complex; an asterisk marks the cross-linked and subsequently DNase I-treated complex; a number sign indicates UV cross-linked complexes after dephosphorylation. Numbers at the left figure indicate the position of molecular size markers in kilodaltons. 4B: Septamer-binding complexes were separated in the first dimension by EMSA followed by UV cross-linking the probe to the core-binding protein. The filled circle indicates a 32 kDa core DNA-binding protein, which is identical in P, N, and G complexes (lanes 2-4, 6, filled circles; also see A). A diamond indicates a protein of 16 kDa, likely the monomeric unit of the DNA-binding protein. Chemical cross-linking enhances the dimerization of this protein (lane 6). An arrowhead indicates the N complex of 84 kDa (lane 7), from which the size of the N-specific protein can be calculated as 29 kDa. A square marks the G-specific complex (lane 8; 78 kDa) from which the size of the G-specific protein has been calculated to be 23 kDa. Molecular weight markers arc in lane 9.

The identical appearance of UV cross-linked DNA-protein complexes in both E14 and E16 striatum (See FIG. 4A) sharply contrasts with the distinct mobility of P, N, and G complexes detected by EMSA (See FIG. 2, compare A, C). The identity of proteins forming the various complexes was investigated by a novel combination of EMSA-mediated chemical and UV cross-linking experiments. EMSA-mediated UV cross-linking experiments confirmed that the basic DNA-binding protein in all three complexes (P, N, and G) is 32 kDa (See FIG. 4B, EMSA I). In addition, a smaller protein-DNA complex was also detected in all three (P-, N-, and G-) bands (See FIG. 4B, EMSA I, diamond, lanes 2-4 and markers) after UV cross-linking. The deduced size of this smaller protein is 16 kDa, half the size of the larger protein (32 kDa). The 16 kDa protein most likely represents the monomer unit of the septamer-binding protein, because after chemical cross-linking, the smaller form was undetectable (See FIG. 4B, EMSA II, lanes 6-8). Loss of the 16 kDa monomeric form would occur as the equilibrium shifts from monomer to dimer in the presence of chemical cross-linker, and in the P-band only the 32 kDa form was detectable after chemical cross-linking (See FIG. 4B, lane 6, filled circle). The 16 kDa protein was named p-sept. Performing a combination of chemical and UV cross-linking on the P-, N-, and G-bands that were excised after EMSA resulted in the identification of additional proteins (See FIG. 4B, EMSA II, lanes 7, 8). The combination of chemical and UV cross-linking of the excised N-band identified an 84 kDa complex (filled triangle). After deducting 32 kDa for the homodimerized p-sept and 22 kDa for the free DNA probe from 84 kDa, the size of the N-specific protein was calculated to be 29 kDa (See FIG. 4B, molecular size panel). This protein was named n-sept. Similar calculations indicated that the size of the G-specific protein (filled rectangle) is 23 kDa (See FIG. 4B, lane 8). This protein was named g-sept. Similar procedures with the excised P-band resulted only in the detection of the 32 kDa p-sept homodimer (See FIG. 4B, lane 6, filled circle), but no additional proteins were apparent. The molecular size of p-sept was independently confirmed by Southwestern analysis using nuclear proteins derived from E15.5 striatum.

The various Sept complexes bend the DNA in a lineage-specific manner:

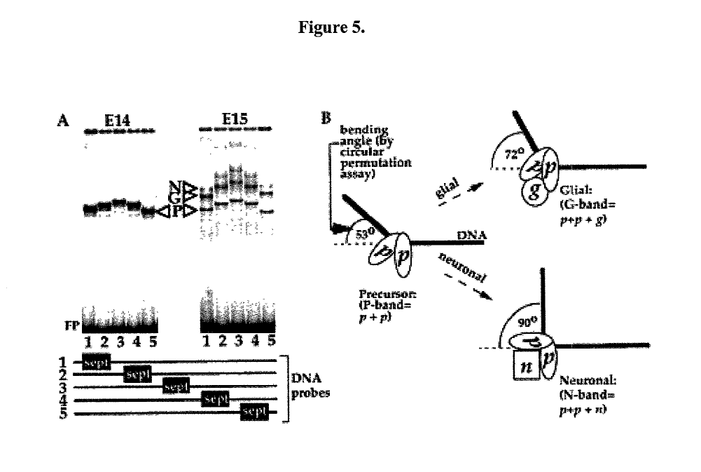

Referring to FIG. 5, depicts distinct bending effects of the various septamer binding protein complexes. 5A: In a circular permutation assay five radioactively labeled nucleic acid fragments contain the septamer element at the various positions relative to fr.C derived from pBENDrENK (probes 1-5, bottom panel). All five fragments were tested with proteins present at E14 (P) and E15 (P, G, N). Different extents of DNA bending are shown as migrational differences of protein-DNA complexes using probes 1-5. FP, Migration of the free probe. The box indicates the relative position of the septamer element within the fragment. 5B: Diagram summarizing the protein components that bind to the septamer motif and bend the DNA in lineage-dependent manner that can regulate gene expression. The P-band observed by EMSA in primordial brain regions (Also see FIGS. 2A,C) is composed of the homodimerized 16 kDa p-sept protein (Also see FIG. 4B). The G-band associated with glial precursors also contains the homodimerized p-sept as the DNA binding domain to which the g-sept of 23 kDa in molecular weight binds (Also see FIG. 4B). Together they form a 55 kDa multimer detected by EMSA as G-band. The DNA binding unit of the N-band associated with neuronal precursors is also composed of the homodimerized p-sept to which a 29 kDa protein, n-sept, binds (also see FIG. 4B). Together they form the 62 kDa multimer detected as N-band by EMSA.

Using a circular permutation assay can address the question of whether the distinct Sept-binding protein complexes can specifically affect DNA structure as measured by DNA bending. As the DNA changes its linear shape to a more “V”-shape configuration, so does the migration of the complex. A bending site closer to the center of the probe results in a higher degree of change in the overall shape of the DNA than a bending site closer to either end. The resulting change in the overall shape of the complex can be detected by electrophoretic mobility shift assay; furthermore, the bending angles can be estimated from the electrophoretic migration differences. The circular permutation assay indicated that septamer-binding proteins indeed bend the DNA on DNA-protein complex formation (See FIG. 5A). Moreover, the bending angle was distinct for each of the lineage-specific complexes (P, G, and N). The P-binding protein(s) present at E14 bent the DNA for 53+4° (See FIG. 5B). The formation of G complex (made of two P proteins plus the G-specific protein) resulted in an increased bending angle up to 72±3° of the DNA. The N complex (the addition of the N protein to the P homodimer) made the DNA bend even more, with a bending angle of 90±2°.

The approach to elucidate the transcriptional controls underlying neural differentiation using the ENK gene as a model system has yielded the following findings: (1) the identification of septamer, a novel DNA motif present within the regulatory regions of some neuronal- and glial-specific genes; (2) the functional indication from “decoy” experiments that septamer proteins act as positive regulators during neurodifferentiation; (3) the biochemical characterization of three distinct nuclear proteins (p-sept, n-sept, and g-sept) that form distinct septamer DNA-containing complexes specific to cells of the progenitor, neuronal and glial lineages; and (4) the demonstration that the distinct septameric protein complexes bend the DNA in a lineage-specific manner that can significantly contribute to the regulatory action in the developing CNS.

The binding site was named septamer because it contains seven nucleotides in the core sequence for specific binding (TTTGCAT) (SEQ ID NO:1). The site is similar to octamer elements, such as the Oct-1 motif (ATTTGCAT) (SEQ ID NO:20) present within the regulatory regions of large numbers of genes with diverse functions (Rosenfeld, 1991; Verrijzer and Van der Vliet, 1993). Of particular note of the Oct-1 motif is the crucial requirement of the 5′ purine nucleotide (A, see above boldface and underlined) for octamer binding (Verrijzer and Van der Vliet, 1993), which is not needed for septamer binding (See FIG. 1B). This level of binding specificity is not without precedent (Ryoo and Mann, 1999), because sequences flanking the core-binding site may contribute to binding specificity (Saade et al., 1997; Swanson and Yang, 1999). Proteins of the Brn family are predominantly expressed in the developing mammalian CNS, but their recognition site is distinct from the oct-1 motif (Wegner et al., 1993); on the other hand, Oct-1 and Oct-2 are not expressed in the brain (Wegner et al., 1993; Eraly et al., 1998). importantly, p-sept that directly binds to the DNA does not bind to the octamer motif (See FIG. 1C). Moreover, sept proteins were not recognized by any of the different polyclonal anti-Oct-1 and Oct-2 antibodies. These findings, along with the estimated molecular mass for the various septamer proteins, indicate that these are distinct from any known POU and octamer-binding proteins (Wegner et al., 1993).

The septamer element has been found within the regulatory regions of genes encoding neurotransmitters, transporters, receptors, surface molecule, and neuron-specific cytoskeletal elements (See Table 1). The list most likely is incomplete because of the low number of GenBank entries for regulatory DNA regions. Therefore septamer-binding proteins can be found on many more neuronal- and glial-specific genes. Functional studies were performed using a septamer-containing DNA competitor molecule (also known as decoy DNA, as described herein), which permits experiments before the availability of antisense molecules (Clusel et al., 1995; Morishita et al., 1998). These studies show that septamer interactions are required for the developmental expression of the tested neuronal- and Glial cells-specific genes and that septamers are likely positive regulators of neurodifferentiation events. Two of the three markers tested in these studies (ENK and NF68) are known to contain septamer motif (See Table 1). However, because of limited GenBank entrees, the presence of the septamer element on the GFAP gene is yet to be determined.

Progenitors populating distinct brain primordia express the 16 kDa p-sept that is homodimerized and forms the P-band as observed by EMSA (See FIGS. 2A,C, 4A,B). Additional evidence for the association of P-band with progenitors was provided by manipulating the differentiation of progenitors in culture (Johe et al., 1996). When E16 striatal cultures were treated with bFGF for 6 d, progenitors persisted, as indicated by P-band, nestin, and cycD2 mRNA expression. None of these markers could be detected in age-equivalent striatum (E22) or in cultures without bFGF treatment. After withdrawal of bFGF, cells differentiated into the astroglial fate (marked by G-band and GFAP mRNA), in agreement with previous studies showing that the default differentiation pathway is astroglial (Craig et al., 1996; Johe et al., 1996). During early postnatal age the P-band was also detected in the cerebellum (Bayer and Altman, 1995). Because postnatal neuro-genesis in the cerebellum gives rise to granule cells, this may explain the presence of the P-band in this period. The N-band was missing at these times; that, however, would not be the first molecular difference between granule cells of the cerebellum and other brain regions (Osborne et al., 1993; Salinas et al., 1994; Dahlstrand et al., 1995).

In the adult brain, the P-band was detected only in the hippocampus and olfactory bulb (See FIGS. 2A,D). This finding agrees with previous descriptions of neural progenitor cell populations in these regions of the adult mammalian CNS (Calof, 1995; McKay, 1997; Gage, 1998; Luskin, 1998). The adult subventricular zone was not examined because of the limited amounts of tissue available.

The appearance and disappearance of the P-, N-, and G-bands were closely correlated with the known developmental gradient of neurogenesis and gliogenesis and differentiation in ontogenetically distinct brain regions (Jacobson, 1993; Bayer and Altman, 1995; see FIG. 2A). For example, both N- and G-bands appeared first in the medulla oblongata (E13-E14) and were not detectable postnatally, indicating the early cessation of neurogenesis and gliogenesis and differentiation in this part of the CNS. Further evidence documenting the association of N- and G-bands with differentiating neurons and Glial cells was obtained by blocking cellular proliferation in embryonic forebrain cultures. Neurons and Glial cells in the mammalian CNS are generated at different time points of development. Accordingly, in the E16 rat forebrain the majority of neurons are already postmitotic, but gliogenesis and differentiation are just on the rise (Jacobson, 1993; Bayer and Altman, 1995). A mitotic blocker applied at developmental age E16 consequently did not have a major effect on the abundance of N-band and the corresponding NF68 mRNA expression but substantially blocked gliogenesis, as measured by a drop in the abundance of G-band and GFAP mRNA. The incomplete elimination of the G-band and GFAP mRNA can be explained by the fact that some progenitors had already differentiated into Glial cells before antimitotic treatment. Positive association of the G-band with Glial cells was demonstrated by analyzing primary cultures of cortical astrocytes, in which only the G-band and GFAP mRNA were present (See FIG. 2E). G-band was also detected in the nuclear extracts of primary cultured oligodendrocytes.

By using a novel combination of UV cross-linking (Tang et al., 1997) and chemical cross-linking (Kom et al., 1972), all three septamer complexes (P-, N-, and G-bands) share an identical DNA-binding unit. This unit is formed by the homodimerized p-sept (See FIGS. 4A,B). However, the available data are insufficient to determine whether homodimerization of p-sept is a prerequisite for DNA binding. As cross-linking studies demonstrated, p-sept continued to be expressed in differentiating neuronal and glial cells. These studies also demonstrated that differentiating neurons express a 29 kDa protein (n-sept), whereas Glial cells express a 23 kDa protein (g-sept). Binding of the 29 kDa n-sept to the p-sept homodimer resulted in the formation of a 62 kDa neuron-specific multimer observed as N-band. Likewise, binding of the 23 kDa g-sept to the homodimerized p-sept resulted in the formation of the Glial cells-specific multimer of 56 kDa observed as G-band (See FIG. 5).

Sept-binding proteins may exert their regulatory effects through altering DNA structure (Spana and Corces, 1990). For example, the binding of the N complex to its DNA site resulted in DNA bending of 90° (See FIGS. 5A,B). This can be permissive for far-distant protein-DNA interactions (Tjian, 1996). These interactions are critical in cellular differentiation as demonstrated in various cells (Grosschedl et al., 1994; Perez et al., 1994; Armstrong et al., 1995; Dyer et al., 1996). The significantly lesser degree of DNA bending (72°) caused by the G complex creates a different structural environment for far-distant DNA-protein interactions. This could be less permissive for far-distant interactions.

The role of the various septamer-binding proteins in regulating the expression of septamer motif-bearing genes (See Table 1) maybe similar to the regulation of Ig gene expression in the B-cell lineage. The B-cell-specific coactivator OCA-B/OBF-1/BOB-1 is expressed in differentiating B-cells and binds to the Oct-1 protein that directly binds to the octamer motif (Luo et al., 1992; Wirth et al., 1995; Cepek et al., 1996; Kim et al., 1996; Knoepfel et al., 1996; Matthias 1998). As suggested by the inactivation of the OCA-B/OBF-1/BOB-1 gene, a precise, lineage-specific assembly of octamer-binding protein complexes is critical in regulating cell-specific expression of Ig genes (Nielsen et al., 1996; Schubart et al., 1996). Similarly, the p-sept-positive multi-potent neuroepithelial progenitor cells will differentiate into either the neuronal or the glial fate depending on the expression of either the n-sept or the g-sept proteins. Thus, septamer-binding proteins contribute to the coordinated expression of septamer motif-bearing genes in regulating the emergence of cellular diversity in the developing nervous system.

Another embodiment of the invention is directed to methods for altering the binding of decoy molecules to proteins by contact with antisense nucleic acids. Antisense nucleic acids can be used to prevent or direct interaction between decoy molecules and proteins, or to prevent expression of certain binding proteins. Methods for creating and incorporating antisense nucleic acids are well know to those of ordinary skill (e.g. see U.S. Pat. Nos. 6,310,047 and 5,087,617)) and can also be applied herein for purposes of interfering with interaction. Further, combinations of antisense and decoy molecules can be used to selectively regulate or direct alteration of cellular functions such as differentiation. For example, by contacting a cell with an antisense against a particular protein to prevent its expression, another nucleic acid binding protein can be directed to carry out a decoy molecule and thereby direct differentiation, or another cellular function associated with decoy-protein interaction.

Definitions:

“Activators,” “inhibitors,” “modulators” and “regulators” refer to molecules that activate, inhibit, modulate and/or regulate an identified function. For example, referring to septamer activity, such molecules may be identified using in vitro and in vivo assays of septamer binding proteins. Inhibitors are compounds, preferably proteins or nucleic acids, that partially or totally alter “septamer” activity, decrease, prevent, or delay septamer activation, or desensitize cellular response to septamers. This may be accomplished by binding to a septamer-regulated proteins directly or via other intermediate molecules. An antagonist of “septamer” is considered to be such an inhibitor. Activators are compounds that may bind to a septamer directly or via other intermediate molecules, thereby increasing or enhancing its activity, stimulating or accelerating its activation, or sensitizing its cellular response. An agonist of a septamer may be considered to be such an activator. A modulator can be an inhibitor or activator, and may or may not bind to the septamer directly but it affect or change the activity or activation of the septamer or the cellular sensitivity to septamer. A modulator also may be a compound such as a small molecule, that inhibits expression of septamer mRNA.

The group of activators, inhibitors, modulators and regulators of this invention also includes genetically modified versions of “septamers” such as, for example, versions with altered activity. The group thus is inclusive of the naturally occurring protein as well as agonists, antagonists, antibodies, small chemical molecules, synthetic ligands, and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, γ-carboxyglutamate, and O-phosphoserine, phosphotheorine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amplicon” generally refers to an amplification product containing one or more genes, which can be isolated from a cell or a tissue. In general, an amplicon is the result of amplification, duplication, multiplication, or multiple expression of nucleic acids or a gene, in vivo or in vitro. “Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kD) and one “heavy” chain (about 0-70 kD). Antibodies exist, for example, as intact immunoglobulins or as a number of well-characterized fragments produced by digestion with various peptidases. While various antibody fragments are defined in terms of the digestion of an intact antibody, one of skilled in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al J Mol Biol. 2000 296:57-86; McCafferty et al., Nature 348:2-4 (1990)), for example. For preparation of antibodies—recombinant, monoclonal, or polyclonal antibodies—any technique known in the art can be used in this invention (see, for example, Kohler & Milstein, Nature 26:49-497 (1997); Kozbor et al., Immunology Today 4: 72 (1983); Cole et al., pp. 77-96 in Monoclonal Antibodies and Cancer Therapy, Alan R. Liss, Inc. (1998)).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology can also be used to identify antibodies and heteromeric Fab fragments that specifically bind to selected antigens (see, for example, McCafferty et al., Nature 348:2-4 (1990); Marks et al., Biotechnology:779-783 (1992)).

“Assays for activators, inhibitors, modulators and/or regulators” refer to experimental procedures including, preferably, expressing p-sept in vitro, in cells, applying putative inhibitor, activator, or modulator compounds, and then determining the functional effects on p-sept activity, as described above. Samples that contain or are suspected of containing p-sept are treated with a potential activator, inhibitor, or modulator. The extent of activation, inhibition, or change is examined by comparing the activity measurement from the samples of interest to control samples. A threshold level is established to assess activation or inhibition. For example, inhibition of a p-sept polypeptide is considered achieved when the p-sept activity value relative to the control is 80% or lower. Similarly, activation of a p-sept polypeptide is considered achieved when the p-sept activity value relative to the control is two or more fold higher.

A “cloning vector” is generally a nucleic acid molecule, preferably, a plasmid, cosmid, or bacteriophage, that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed with the cloning vector. Marker genes include genes that provide antibiotic resistance, preferably, tetracycline resistance or ampicillin resistance.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and TGG (tryptophan), can be modified conservatively to yield a functionally-identical peptide or protein molecule.

As to amino acid sequences, one skilled in the art will recognize that substitutions, deletions, or additions to a polypeptide or protein sequence which alter, add or delete a single amino acid or a small number (typically less than ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine.

The term “decoy molecule” refers to a nucleic molecule, which may be single or double stranded, that comprise DNA, RNA, or PNA, and that contains a sequence of a protein binding site, preferably a binding site for a regulatory protein and more preferably a binding site for a transcription factor.

The term “differentiation marker” refers to protein, carbohydrate or nucleic acid molecules whose presence, absence, or level of expression or state (bound verses unbound) is indicative of the differentiation status of a cell or cell line. Preferred differentiation markers include, but are not limited to, mRNA or proteins expressed from the genes p-sept, g-sept, n-sept, nestin or Cyclin D2.

The term “differentiation status” refers to the maturation or developmental stage, cell cycle stage, or developmental potential of a cell, preferably a progenitor or stem cell. The differentiation status of many cells can be determined by detecting changes in the expression of or the levels of expression of one or more, or a combination of, differentiation markers.

In general, a “gene” is a region on the genome that is capable of being transcribed to an RNA that either has a regulatory function, a catalytic function, and/or encodes a protein. A gene typically has introns and exons, which may organize to produce different RNA splice variants that encode alternative versions of a mature protein. The skilled artisan will appreciate that the present invention encompasses all septamer-binding proteins-encoding transcripts that may be found, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene, can be any portion from the gene, which may or may not represent a functional domain, for example, a DNA binding domain, etc. A fragment may preferably include nucleotide sequences of at least 5 contiguous nucleic acids, and preferably at least about 6, 7, or more contiguous nucleic acids or any integer thereabout.

The term “expression” generally refers to the biosynthesis of a gene product. For example, in the case of a structural gene, expression involves transcription of the structural gene into mRNA and the translation of mRNA into one or more polypeptides.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers. Such a gene is said to be “operably linked to” or “operatively linked to” the regulatory elements, which means that the regulatory elements control the expression of the gene.

Also included in the invention are “functional polypeptides,” which possess one or more of the biological functions or activities of a protein or polypeptide of the invention. These functions or activities include the ability to bind some or all of the proteins which normally bind to a nuclear protein, for example, p-sept protein.

The functional polypeptides may contain a primary amino acid sequence that has been modified from that considered to be a standard sequence of nuclear proteins, for example, p-sept, described herein. Preferably these modifications are conservative amino acid substitutions, as described herein.

A “host cell” is a naturally occurring cell or a transformed cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, CHO, HeLa, and the like.

The term “immunoassay” is an assay that utilizes the binding interaction between an antibody and an antigen. Typically, an immunoassay uses the specific binding properties of a particular antibody to isolate, target, and/or quantify the antigen.

The terms “isolated,” “purified,” or “biologically pure” refer to material that is free to varying degrees from other components which normally accompany the specific component being isolated or purified as found in its native, artificial or other state. “Isolate” denotes any degree of separation. “Purify” denotes a degree of separation that is higher than isolation. A “purified” or “biologically pure” protein is typically sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. Various levels of purity may be applied as needed according to this invention in the different methodologies set forth herein; the customary purity standards known in the art may be used if no standard is otherwise specified.

A “label” or a “detectable moiety” is generally a composition that when linked with the nucleic acid or protein molecule of interest renders the latter detectable. Detectability maybe via spectroscopic, photochemical, biochemical, immunochemical, or chemical means, or any means known to those of ordinary skill in the art. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A “labeled nucleic acid or oligonucleotide probe” is one that is bound, either covalently, through a linker or a chemical bond, or noncovalently, through ionic, van der Waals, electrostatic, hydrophobic interactions, or hydrogen bonds, to a label such that the presence of the nucleic acid or probe may be detected by detecting the presence of the label bound to the nucleic acid or probe.

“Nucleic acid” refers to deoxyribonucleotides or ribonucleotides and polymers thereof in either single- or double-stranded form. The term encompasses nucleic acids containing known nucleotide analogs or derivatives, or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res. 19:081 (1991); Ohtsuka et al, J. Biol. Chem. 260:260-2608 (198); Rossolini et al., Mol. Cell. Probes 8:91-98 (1994)). The term nucleic acid is used interchangeably with gene, cDNA, mRNA, oligonucleotide, and polynucleotide.

As used herein a “nucleic acid probe or oligonucleotide probe” is generally defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The term “operably linked” is used to describe the connection between regulatory elements and a gene or its coding region. That is, gene expression is typically placed under the functional control of certain regulatory elements, including constitutive or inducible promoters, tissue-specific regulatory elements, and enhancers. Such a gene is said to be “operably linked to” or “operatively linked to” the regulatory elements.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides of the invention can be chemically synthesized or synthesized by recombinant DNA methods; or, they can be purified from tissues in which they are naturally expressed, according to standard biochemical methods of purification.

A “recombinant host” is generally any prokaryotic or eukaryotic cell or cells that contain either a cloning vector or expression vector. This term also includes those prokaryotic or eukaryotic cells that have been genetically engineered to contain the cloned gene(s) in the chromosome or genome of the host cell.

In eukaryotes, RNA polymerase II catalyzes the transcription of a structural gene to produce mRNA. A DNA molecule can be designed to contain an RNA polymerase II template in which the RNA transcript has a sequence that is complementary to that of a preferred mRNA. That RNA transcript is termed an “antisense RNA”. Antisense RNA molecules can inhibit transcription and/or translation.

Antisense molecules, as described here, can be used to block, inhibit, or alter septamer function or septamer downstream function; the protein-nucleic acid interaction; or the binding of septamer to cellular protein. Antisense molecule can also block, inhibit, or alter the interaction of a septamer or a nucleic acid decoy molecule and a nuclear protein; protein expression; and differentiation of a progenitor or stem cell.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

The term “septamer” refers to a nucleic acid molecule comprising the seven-mer polynucleotide sequence TTTGCAT (SEQ ID NO:1). This sequence is a novel regulatory element found in a variety of neuronal cell, glial cell, and other cellular genes including, but not limited to, the genes which encode enkephalin (ENK), neuronal cell adhesion molecule, neurofilament of 68 kDa (NF68), growth-associated protein of 43 kDa, glial high-affinity glutamine transporter, and tyrosine hydroxylase.

The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at TR, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary, non-limiting stringent hybridization conditions can be as following: 50% formamide, 5×SSC, and 1% SDS, incubating at 42° C., or, 5×SSC, 1 SDS, incubating at 65° C., with wash in 0.2×SSC, and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The invention is further described by the following examples, which do not limit the invention in any manner.

### EXAMPLES

### Example 1

### Microdissection of Rat Brain and Preparation of Nuclear Extracts

Dissection of the rat brain into various ontogenetically and phenotypically distinct brain regions between embryonic stages E10-E21 and postnatal ages P2-P28 as well as the preparation and characterization of nuclear extracts from the dissected brain regions, peripheral tissues, and cultured cells are performed as described (Dobi et al., 1997). The broad-range protease inhibitor [4-(2-aminoethyl)-benzenesulfonyl fluoride hydrochloride] (AEBSF) is used throughout the preparation of nuclear extracts and the subsequent steps. HeLa cell nuclear extracts are purchased from Stratagene (La Jolla, Calif.) and CellTech (Minneapolis, Minn.).

### Example 2

### Generation of Restriction DNA Fragments and Synthetic Oligonucleotides

A DNA fragment corresponding to nucleotides −542 to −384 of the rat ENK (rENK) gene is generated (Dobi et al., 1997) from the plasmid pRESS1 (Joshi and Sabol, 1991) by PCR using sequence-specific primers and cloned into a pCRII plasmid vector (Invitrogen, Carlsbad, Calif.). For binding studies, the 160 bp fragment rENK-542;-379 from the plasmid (EcoRI digest), is labeled by [α-³²P]dATP incorporation with Klenow polymerase, and purified on 4% polyacryamide gel (acrylamide/bisacrylamide, 40:1) in 1×Tris acetate-EDTA buffer. The DNA fragment is excised from the gel and eluted. Ten femtomoles of DNA probe are used per EMSA, as known in the art. Partly overlapping subfragments are designed based on the consensus sequence information available for the rENK gene (Durkin et al., 1992). A subfragment (rENK-496;-467) 5′-AAATATTGGTTTGCATAATCATTGACTGCC-3′ (SEQ ID NO:2) retains all the binding activity and therefore is used in subsequent studies (underlined sequence is septamer). Synthetic oligonucleotides are radioactively labeled by [γ-³²P]dATP and polynucleotide kinase, annealed to the complementary strand, and gel-purified as described (Dobi et al., 1995a). The synthetic oligonucleotides are also used in DNA affinity chromatography.

### Example 3

### Electrophoretic Mobility Shift Assay (EMSA)

Binding reactions are performed as described (Dobi et al., 1995a). Briefly, 10 fmol of rENK-542;-379 or the synthetic probe rENK-496;-467 (See FIG. 1) is added to 3 μg of nuclear extracts in the presence of 0.2 mg/ml poly(dIdC) (Boehringer Mannheim GmbH, Heidelberg, Germany) in binding buffer (12 mM HEPES, pH 7.9, 60 mM KCl, 1 MM MgCl₂, 0.2 mM EDTA, 0.5 mM DTT, 0.1 mM AEBSF, and 5% glycerol) in a total volume of 10 μl. The reaction mixture is incubated at 25° C. for 20 min, and the complex separated from the free probe by electrophoresis on a 4% polyacrylamide gel (acrylamide/bisacrylamide, 40:1) in 1×Tris borate-EDTA (TBE) buffer at 12 W of constant power for 2 hr at room temperature. Gels are fixed in 10% acetic acid, transferred to Whatman (Maidstone, UK) 3MM paper, dried, subjected to autoradiography, and analyzed in a PhosphorImager (Molecular Dynamics, Sunnyvale, Calif.). In the competition assay, 1 pmol of unlabeled competitor DNA is added to the reaction mixture before addition of the nuclear extract, resulting in a 100-fold excess of the competitor DNA.

### Example 4

### Methylation Interference Assay

Radioactively labeled rENK-542;-379 (See above) is further cut by BsmAI restriction endonuclease and gel-purified. The G residues are methylated by dimethyl sulfate using the Maxam-Gilbert sequencing kit according to the manufacturer's instructions (DuPont, Boston, Mass.). Eighty femtomoles of probe (6×10⁶ cpm/pmol) are combined with 40 μg of nuclear extracts derived from E16 striatum in the presence of 15 μg of poly(dIdC) in binding buffer (See above) in a total volume of 100 μl. The optimal amount of protein used can be determined by titration. The mixture is incubated at room temperature for 30 min. The bound and free fractions are separated on a 4% polyacrylamide gel as above. The gel is autoradiographed, and subsequently the bound and free fractions excised and eluted by soaking overnight at 37° C. in 400 μl of elution buffer (0.5 M NH₄OAc, 0.1% SDS, 2 mM EDTA, and 10% methanol). The eluted probes are extracted with phenol/chloroform and precipitated with ethanol twice in the presence of 10 μg of glycogen. The pellets are washed with 70% ethanol, dried, and subjected to chemical cleavage (Maxam and Gilbert, 1977). The DNA is lyophilized twice and dissolved in H₂O, and the activity adjusted to 2000 cpm/μl. Three microliters of samples are mixed with 3 μl of 90% formamide and loading dyes, heated to 95° C. for 3 min. Three thousand counts per minute of sample are analyzed in each lane of a 6% polyacrylamide, 7 M urea, TBE sequencing gel. After the separation, the gel is exposed to x-ray film at −70° C. for 72 hr. The identity of the core binding site, designated septamer motif, is independently identified using EMSA in combination with the left-to-right and right-to-left truncated probe strategy (Dobi et al., 1995b).

### Example 5

### Antibody Supershift Assay

Antibodies raised against Oct-1 (L. Staudt, National Institute of Diabetes and Digestive and Kidney Diseases, National Institutes of Health, Bethesda, Md.) and Oct-2 proteins (Santa Cruz Biotechnology, Santa Cruz, Calif.) are tested under two conditions: (1) 3 μg of nuclear extracts incubated with 10 ng of either anti-Oct-1 rabbit polyclonal IgG or anti-Oct-2 rabbit polyclonal IgG in binding buffer (See above) in the presence of 10 μg of BSA and 0.2 mg/ml of poly(dIdC) at 0° C. for 40 min in a total volume of 10 μl; 10 fmol of radioactively labeled rENK-542;-379 probe are added to the reaction mixture and incubated at room temperature for an additional 30 min. (“pre”); and (2) in a second set of experiments, 10 fmol of the radioactively labeled probe are first incubated with 3 μg of nuclear extracts as above, followed by addition of 10 ng of either anti-Oct-1 rabbit polyclonal IgG or anti-Oct-2 rabbit polyclonal IgG; the reaction mixtures are incubated at 4° C. for 40 min. In control reactions, 3 μg of HeLa nuclear extract is tested under identical conditions. Complexes are separated and analyzed by EMSA as above.

### Example 6

### Cell Cultures

A. Astrocytes. Cortical astrocytes are obtained from 1-day-old neonatal (Sprague Dawley) rats as described (McCarthy and de Vellis, 1980). After replating confluent cells, astrocytes are grown in DMEM with high glucose and 20 mM HEPES, pH 7.4, containing 10% fetal bovine serum. Cells are harvested and nuclear proteins and cytoplasmic total RNA are prepared as previously described (Dobi et al., 1995a).

B. Expanding cultures of striatal progenitors. Cells derived from E16 striatum are isolated and cultured in the presence of 10 ng/ml basic fibroblast growth factor (bFGF) as mitogen as described (Johe et al., 1996). After 6 d of mitotic expansion in the presence of bFGF, differentiation is initiated by removing bFGF and culturing cells in serum-free medium. Some cultures are harvested at the end of the expansion period (+bFGF); others are harvested 3 d after withdrawal of the mitogen (bFGF withdrawn). Cell culture medium is removed by aspiration; cultures are washed with 1×PBS, and after removal of PBS culture dishes are frozen on dry ice. Nuclear extracts are prepared using the microprocedure adopted for cultured cells, and total cellular RNA is prepared from cytoplasmic supernatants as described (Dobi et al., 1995a).

C. Differentiating neural cultures of rat embryonic forebrain. Forebrains are dissected from E15.5 embryos and collected in ice-cold D1 solution as previously described (Agoston et al., 1991). After several steps of washing with equilibrated Earl's balanced salt solution the tissue is minced with sterile blades and dissociated by using the papain dissociation system according to the manufacturer's instructions (Worthington, Lakewood, N.J.). The resulting cell suspension is diluted with equilibrated MEM containing 10% bovine serum, 10% heat-inactivated horse serum, N3 nutrient mixture (Brenneman et al., 1987), 2.5 mM glutamine, and anibiotic-antimycotic mixture (Life Technologies, Gaithersburg, Md.), and the number of viable cells determined by the trypan blue exclusion technique. Cells are plated at the density of 400,000/ml in poly-L-lysine-precoated tissue culture flasks. Medium is changed 12 hr after plating, corresponding to 0.5 d in vitro (DIV), to MEM containing 5% heat-inactivated horse serum, N3, 2.5 mM glutamine, and antibiotic-antimycotic mixture (Life Technologies). Some cultures receive a mixture of 5′-fluoro-2′-deoxyuridine and uridine (FUDR) at final concentrations of 15 and 35 μg/ml, respectively, to block cellular proliferation. At this concentration FUDR completely blocks proliferation without causing death of the mitotically arrested cells (Brenneman et al., 1987). Cultures are harvested at 2.5 DIV by the removal of the medium by aspiration and placing the flasks on dry ice. Nuclear extracts and cytoplasmic supernatants are prepared using the microprocedure as above. Total cytoplasmic RNA is extracted and reverse-transcribed for PCR characterization of gene expression pattern of the cultures as above. As RT-PCR analysis has demonstrated, these culture conditions are permissive for nestin-expressing as well as neurofilament of 68 kDa (NF68)- and glial fibrillary acidic protein (GFAP)-expressing cells.

D. Embryonic striatal cultures for DNA competition assay. Striata are dissected from E17 embryonic rat brains and dissociated by using the papain dissociation system as above. Cells are plated in MEM containing 5% heat-inactivated horse serum, N3, 2.5 mM glutamine, and anibiotic-antimycotic mixture (Life Technologies) at the density of 150,000 cells/ml in poly-L-lysine-precoated 24 well plates. Medium is changed 12 hr after plating.

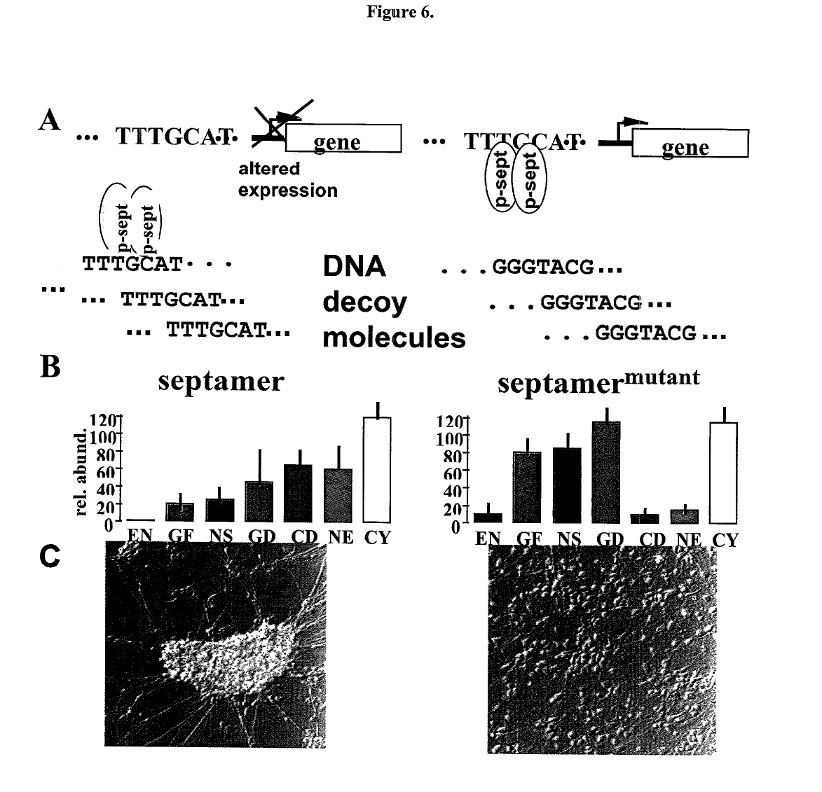

E. DNA competition assay. The assay is performed essentially as described for DNA molecular decoy (Yamashita et al., 1998). As competitor DNA molecule the synthetic DNA fragment rENK-496; -467 previously tested in EMSA is used (See above). For control competitor DNA, a transversion mutant of the septamer DNA element is created (the core TTTGCAT was replaced by GGGTACG (SEQ ID NO:3)), leaving the flanks identical (septamer^{mut}). EMSA shows that the mutated fragment failed to bind nuclear proteins (See FIG. 6). The competitor and control DNA molecules are introduced into cells. Numerous methods can be used, including the polyethyleneimine delivery system (Boussif et al., 1995). Four to 6 hr after transfection, the medium is changed, and cells were cultured as above. Cultures are harvested 2 d after DNA decoy and processed for RNA extraction followed by reverse transcription.

### Example 7

### RNA Preparation, Reverse Transcription, and PCR

Total cellular RNAs are prepared by using the RNeasy technique (Qiagen, Hilden, Germany) according to the manufacturer's instructions. Equal amounts of RNA are reverse-transcribed using oligo-dT priming in the Superscript II system according to the manufacturer's instructions (Life Technologies). PCR conditions and primers for ENK, NF68, GFAP, and cyclophilin primers are as described earlier (Dobi et al., 1995a). Cyclin D2 (cycD2) primers are as published (Freeman et al, 1994). Nestin primers (5′-ACTGAGGATAAGGCAGAGTTGC-3′ (SEQ ID NO:21) and 5′-GAGTCTTGTTCACCTGCTTGG-3′ (SEQ ID NO:22)) are designed using the GeneWorks program (Oxford Molecular, Campbell, Calif.) using the rat nestin gene sequence (GenBank accession number M34384). PCR reactions are performed as described (Dobi et al., 1995a). PCR amplicons are separated on 5-20% acrylamide gels (Novex, San Diego, Calif.) or thin agarose gels (Dobi et al., 1997). Bands are visualized and quantified either in a PhosphorImager (Molecular Dynamics) or in an Eastman Kodak (Rochester, N.Y.) SP700 imaging system after SYBR green-I (Molecular Probes, Eugene, Oreg.) staining.

### Example 8

### UV Cross-Linking in Solution

A synthetic septamer oligonucleotide for UV cross-linking is designed to form a partial hairpin structure with a recessed 3′ end (5′-GTCAATGATTATGCAAACCAATATTTGCGTCAGGCTAGCCTGACG-3′ (SEQ ID NO:23); also see FIG. 1). The purified oligonucleotide is annealed to allow formation of the hairpin in Klenow polymerase buffer (80 mM Tris-HCl, pH 8.3, 25 mM MgCl₂) at room temperature for 10 min. The recessive end is filled in with deoxynucleotides and [α-³²P]dATP using Klenow polymerase. The labeled double-stranded DNA is gel-purified, and ˜30 fmol of probe (2×1⁷ cpm/pmol) combined with 3 μg of nuclear extract from either E14 or E16 rat striatum in the presence of 0.2 μg/μl poly(dIdC) in binding buffer in a total volume of 30 μl and incubated at room temperature for 30 min. The UV cross-linking in solution is performed by exposing the binding reaction mixtures to 0.12 J of UV light for 4 hr at 4° C. Part of the reaction mixture is incubated with 1 U of DNase I (Worthington Biochemical, Freehold, N.J.) at room temperature for 5 min. To remove phosphate groups from the proteins, 0.3 U of protein tyrosine phosphatase 1B and 0.3 U of protein phosphatase type-2A (specific for phosphoseryl and phosphothreonyl proteins; Upstate Biotechnology, Lake Placid, N.Y.) are added to the reaction mixtures, which are then incubated for 15 min at 30° C. All solutions included 0.5 mM of the protease inhibitor AEBSF. The complexes are analyzed on a 4-20% gradient Tris-glycine/SDS gel (Novex).

### Example 9

### Combined Chemical and UV Cross-Linking

A synthetic oligonucleotide containing the septamer element for UV cross-linking is designed to form a partial doublet hairpin structure with a recessive 3′ end and prepared as above. Approximately 10 fmol of probe (2×10⁷ cpm/pmol) is combined with 3 mg of nuclear extract from either E14 or E16 striatum in the presence of 0.2 μg/μl of poly(dIdC) in binding buffer (See above) in a total volume of 10 μl and incubated at room temperature for 30 min. The DNA-protein complex is separated from the free probe on a 4% polyacrylamide gel in TBE buffer. After electrophoresis, the gel is laid on a plastic wrap and slid onto a previously developed x-ray film and transferred onto the top of a ready pack X-Omat AR film (Kodak) along with position markers. The gel-film sandwich is put in a UV Stratalinker oven (Stratagene) and covered with a thin layer of 1% glutaraldehyde (Sigma, St. Louis, Mo.) where indicated. The optimal concentration of glutaraldehyde is determined by titration. The UV cross-linking is performed with 0.12 J of energy for 4 hr at 4° C. so that exposure to the x-ray film, the DNA-protein cross-linking with UV light, and the protein-protein cross-linking with glutaraldehyde are performed simultaneously. The positions of the DNA-protein complexes and free DNA are identified after developing the film. The DNA-protein complexes and the free probe are excised from the gel and analyzed on 4-20% gradient Tris-glycine/SDS gel (Novex).

### Example 10

### Circular Permutation Assay

A 26 mer synthetic oligonucleotide containing the septamer element along with flanks (-467; -496 bp of the ENK gene; See FIG. 1) is inserted into the pBend5 plasmid (Adhya and Kim, National Cancer Institute, National Institutes of Health, Bethesda, Md.) using XhaI and SalI restriction sites. The resulted pBend5/sept plasmid is cut by NruI, EcoRV, BamHI, ClaI, and MluI restriction endonucleases. The resulting 146-bp-long fragments are radioactively labeled at the ends by polynucleotide kinase (Boehringer Mannheim, Indianapolis, Ind.) and [γ-³²P]ATP (Amersham, Arlington Heights, Ill.) and gel-purified. Reaction mixtures for DNA-protein binding reactions are prepared as described above, except that a 5 fmol labeled fragment is combined with 1 μg of nuclear extracts derived from either E14 or E15 striatum. The electrophoretic separation is performed in 8% polyacrylamide gel (acrylamide/bis, 40:1, w/w) in 1×TBE buffer under 13 W of constant power. After separation the gel is fixed, dried, and subjected to autoradiography. To estimate the DNA bending angles, the relative migration values are measured, and the approximate “α” values calculated as described (Kim et al., 1989). If the mobilities of the free probes show only insignificant variations, corrections for free probe mobility differences are unnecessary.

Other embodiments and uses of the invention will be apparent to those skilled in the art from consideration of the specification and practice of the invention disclosed herein. All references cited herein for any reason, including all U.S. and foreign patents and patent applications, are specifically and entirely incorporated by reference. It is intended that the specification and examples be considered exemplary only, with the true scope and spirit of the invention indicated by the following claims.

[image]

[image]

[image]

[image]

[image]

[image]

1. A method for altering the differentiation status of a mammalian cell comprising:
contacting a nucleic acid molecule containing the sequence set forth in SEQ ID NO:1 or SEQ ID NO:2, with said cell, wherein said molecule is capable of entering the cell; and
culturing said cells whereby the differentiation status of the cell is altered.

2. The method of claim 1, wherein the differentiation status of said cell after culturing indicates that said cell is a neural progenitor cell.

3. The method of claim 1, wherein the nucleic acid contains a base other than A, U or a derivative thereof, at the position immediately 5′ to the sequence of SEQ ID NO:1.

4. The method of claim 1, wherein the mammalian cell is a human cell.

5. The method of claim 1, wherein the differentiation status of the cell is determined by a change in expression level of a differentiation marker.

6. The method of claim 5, wherein the differentiation marker is selected from the group consisting of a p-sept protein, a g-sept protein, an n-sept protein, a nestin protein, a cyc D2 protein, and fragments and combinations thereof.

7. A cell or cell line produced by the method of claim 1.

8. A method for isolating a protein comprising:
contacting biological material containing said protein with a nucleic acid molecule comprising the sequence set forth in SEQ ID NO:1 or SEQ ID NO:2; and
separating protein that binds to said nucleic acid molecule from other components of the extract.

9. The method of claim 8 wherein the nucleic acid molecule is bound to a solid support.

10. An isolated complex comprising a protein that binds to a nucleic acid molecule containing the sequence of SEQ ID NO:1 or SEQ ID NO:2.

11. The complex of claim 10 wherein the protein is selected from the group consisting of a p-sept protein of approximately 16 kDa, a g-sept protein of approximately 23 kDa, an n-sept protein of approximately 29 kDa, and fragments, and combinations thereof.

12. An antibody or antibody fragment that specifically binds to the complex of claim 10.

13. A diagnostic kit comprising the antibody or antibody fragment of claim 12.

14. A method for producing a cell with an altered differentiation status comprising:
contacting a nucleic acid molecule comprising the sequence set forth in SEQ ID NO:1 or SEQ ID NO:2 with said cell wherein the molecule is capable of entering the cell; and
culturing the cell whereby the differentiation status of the cell is altered.

15. The method of claim 14, wherein before culturing the cell has the differentiation status of a stem cell and after culturing the cell has the differentiation status of a neural progenitor cell.

16. The method of claim 14, wherein the molecule contains a base other than A, U or a derivative thereof at the position immediately 5′ to the sequence of SEQ ID NO:1.

17. The method of claim 14, wherein the differentiation status of the cells is determined by a change in the level of a differentiation marker.

18. The method of claim 14, wherein the differentiation marker is selected from the group consisting of a p-sept protein, a g-sept protein, an n-sept protein, a nestin protein, a cyc D2 protein, and fragments and combinations thereof.

19. A cell or cell line produced by the method of claim 14.

20. A method of treating a disease comprising:
contacting a nucleic acid molecule containing the sequence set forth in SEQ ID NO:1 or SEQ ID NO:2 to a cell whereby the differentiation status of said cell is altered which thereby ameliorates a symptom of said disease.

21. The method of claim 20, wherein the cell is a stem cell or a progenitor cell.

22. The method of claim 20, wherein the cell is a neural cell.

23. The method of claim 20, wherein the altered cell generates a homogeneous population of such cells for transplantation.

24. The method of claim 20, wherein the disease is a CNS disorder, a neuro-degenerative disease, or a traumatic brain injury.

25. A pharmaceutical composition comprising: an isolated nucleic acid molecule comprising the sequence set forth in SEQ ID NO:1 or SEQ ID NO:2, wherein the molecule is capable of entering a cell and altering the differentiation status of said cell.

26. The composition of claim 25 further comprising one or more pharmaceutically acceptable carriers.

27. The composition of claim 25, wherein the nucleic acid molecule comprises DNA, RNA or PNA.

28. The composition of claim 25, wherein the cell is a neural cell.

29. The composition of claim 25, wherein the molecule contains a base other than A, U or a derivative thereof at the position immediately 5′ to the sequence of SEQ ID NO:1.

30. The composition of claim 25, wherein the molecule is capable of altering a cellular function associated with septamer activity.

31. The composition of claim 30 wherein the cellular function associated with septamer activity is a differentiation status of the cell.

32. The composition of claim 25 wherein the cell is a neural cell.

33. The composition of claim 32, which is administered to a patient with a neuro-degenerative disease or a traumatic brain injury.
